(19) United States
(12) Patent Application Publication
(10) Pub. No.: US 2015/0166439 A1
(43) Pub. Date: Jun. 18, 2015

(54) INTEGRATION OF MTO WITH ON PURPOSE BUTADIENE

(71) Applicant: UOP LLC, Des Plaines, IL (US)

(72) Inventors: Andrea G. Bozzano, Northbrook, IL (US); Carl J. Stevens, Lake Forest, IL (US); John J. Senetar, Naperville, IL (US)

(21) Appl. No.: 14/566,124

(22) Filed: Dec. 10, 2014

Related U.S. Application Data
(60) Provisional application No. 61/916,769, filed on Dec. 16, 2013.

(51) Int. Cl. C07C 5/333; C07C 5/09; C07C 4/06; C07C 1/22

(52) U.S. Cl. CPC C07C 5/3335; C07C 1/22; C07C 5/09; C07C 4/06; C07C 2523/80

(57) ABSTRACT

A process is presented for the production of 1,3-butadiene. The process include separating C4 olefins from an olefin stream generated by a methanol to olefins conversion reactor. The C4 stream is process and 1,3-butadiene is recovered, The remaining C4 stream is passed to a dehydrogenation unit to generate more butadienes. The dehydrogenation process stream is processed to recover products, such as 1-butene and 1,3-butadiene.

### CROSS REFERENCE TO RELATED APPLICATION

This application claims the benefit of U.S. Provisional Application No. 61/916,769 filed Dec. 16, 2013.

### FIELD OF THE INVENTION

The present invention relates to a process for the production of butadiene. In particular, this is a process for the integration of a butadiene production process into a petrochemical plant.

### BACKGROUND OF THE INVENTION

The use of plastics and rubbers is widespread in today's world. The production of these plastics and rubbers are from the polymerization of monomers which are generally produced from petroleum. The monomers are generated by the breakdown of larger molecules to smaller molecules which can be modified. The monomers are then reacted to generate larger molecules comprising chains of the monomers. An important example of these monomers is light olefins, including ethylene and propylene, which represent a large portion of the worldwide demand in the petrochemical industry. Light olefins, and other monomers, are used in the production of numerous chemical products via polymerization, oligomerization, alkylation and other well-known chemical reactions. Producing large quantities of light olefin material in an economical manner, therefore, is a focus in the petrochemical industry. These monomers are essential building blocks for the modern petrochemical and chemical industries. The main source for these materials in present day refining is the steam cracking of petroleum feeds.

Another important monomer is butadiene. Butadiene is a basic chemical component for the production of a range of synthetic rubbers and polymers, as well as the production of precursor chemicals for the production of other polymers. Examples include homopolymerized products such as polybutadiene rubber (PBR), or copolymerized butadiene with other monomers, such as styrene and acrylonitrile. Butadiene is also used in the production of resins such as acrylonitrile butadiene styrene.

Butadiene is typically recovered as a byproduct from the cracking process, wherein the cracking process produces light olefins such as ethylene and propylene. With the increase in demand for rubbers and polymers having the desired properties of these rubbers, an aim to improving butadiene yields from materials in a petrochemical plant will improve the plant economics.

### SUMMARY OF THE INVENTION

The present invention is directed to a process for the on-demand production of 1,3-butadiene, through the diversion of an olefin stream generated by an oxygenate to olefins conversion process.

A first embodiment of the invention is a process for the production of butadiene comprising passing a process stream from an oxygenate to olefins reactor to a first separation unit to generate a first process stream comprising light olefins, and a second process stream comprising C4 and heavier olefins; passing the second process stream to a second separation unit to generate a C4 process stream and a heavies process stream, wherein the C4 process stream comprises butenes and butanes, and an isobutene content of less than 1 wt %; and passing the butene process stream to a dehydrogenation unit to generate a dehydrogenation process stream comprising butadienes. An embodiment of the invention is one, any or all of prior embodiments in this paragraph up through the first embodiment in this paragraph further comprising passing the dehydrogenation process stream to a butadiene extraction unit to generate a butadiene stream comprising 1,3-butadiene, and a second C4 stream. An embodiment of the invention is one, any or all of prior embodiments in this paragraph up through the first embodiment in this paragraph further comprising passing the second C4 stream to the dehydrogenation unit. An embodiment of the invention is one, any or all of prior embodiments in this paragraph up through the first embodiment in this paragraph further comprising passing the dehydrogenation process stream to a dewatering unit to generate a butadiene stream with reduced water content. An embodiment of the invention is one, any or all of prior embodiments in this paragraph up through the first embodiment in this paragraph further comprising passing the dewatered stream to a degassing unit to generate a degassed stream. An embodiment of the invention is one, any or all of prior embodiments in this paragraph up through the first embodiment in this paragraph wherein the dehydrogenation unit is an oxidative dehydrogenation unit.

A second embodiment of the invention is a process for the production of 1,3 butadiene, comprising passing an oxygenate stream to an oxygenate conversion reactor to generate a process stream comprising olefins, diolefins and oxygenates; passing the process stream to a first separation unit to generate a light olefins stream, an oxygenate recycle stream, a C5+ stream, and a C4 process stream having an isobutene content of less than 1 wt %; passing the C4 stream to a butadiene extraction unit to generate a 1,3 butadiene stream and a second process stream; passing the second process stream to a dehydrogenation unit to generate a dehydrogenated stream; and passing the dehydrogenated stream to the butadiene extraction unit. An embodiment of the invention is one, any or all of prior embodiments in this paragraph up through the second embodiment in this paragraph wherein the dehydrogenation unit is an oxidative dehydrogenation unit. An embodiment of the invention is one, any or all of prior embodiments in this paragraph up through the second embodiment in this paragraph further comprising passing the second process stream to a second separation unit to generate a stream comprising 1-butene, and a second C4 stream. An embodiment of the invention is one, any or all of prior embodiments in this paragraph up through the second embodiment in this paragraph wherein the dehydrogenation unit is a non-oxidative dehydrogenation unit.

A third embodiment of the invention is a process for the production of 1,3-butadiene, comprising passing an oxygenate stream to an oxygenate conversion reactor to generate a first process stream comprising olefins, diolefins and oxygenates; passing the first process stream to a first separation unit to generate a light olefins stream, a C4 stream and a recycle stream; passing the C4 stream to a selective hydrogenation unit to generate a C4 stream with reduced acetylenes content; passing the C4 stream with reduced acetylene content to a butadiene extraction unit to generate a 1,3 butadiene stream, and a residual C4 stream; passing the byproduct C4 stream to a selective hydrogenation unit to generate a hydrogenated raffinate stream; passing the hydrogenated raffinate stream to a butene separation unit to generate an overhead stream comprising 1-butene and a bottoms stream comprising 2-butene; passing the bottoms stream to a dehydrogenation unit to generate a dehydrogenated stream comprising 1,3-butadiene; passing the dehydrogenated stream to a dewatering unit to generate a dewatered dehydrogenated stream; passing the dewatered stream to a light gas stripping unit to generate a light end stream and a second process stream comprising 1,3-butadiene; passing the second process stream to a second separation unit to generate a second overhead stream comprising 1,3-butadiene and a second bottoms stream; and passing the second overhead stream to the butadiene extraction unit. An embodiment of the invention is one, any or all of prior embodiments in this paragraph up through the third embodiment in this paragraph further comprising passing the second bottoms stream to the dehydrogenation unit. An embodiment of the invention is one, any or all of prior embodiments in this paragraph up through the third embodiment in this paragraph further comprising passing the dehydrogenation process stream to a oxygenate removal unit to generate an overhead stream with reduced oxygenate content; and passing the overhead stream with reduced oxygenate content to the butadiene recovery unit. An embodiment of the invention is one, any or all of prior embodiments in this paragraph up through the third embodiment in this paragraph wherein the first separation unit can include operational units for the separation of C5+ hydrocarbons from the first process stream.

A fourth embodiment of the invention is a process for the production of 1,3 butadiene comprising passing an oxygenate stream to a methanol to olefins unit to generate an MTO stream comprising olefins; passing the MTO stream comprising C2+ olefins to a light olefin recovery unit to generate a first stream comprising ethylene, a second stream comprising propylene, a third stream comprising butenes, and a fourth stream comprising C5+ hydrocarbons; passing the third stream to a dehydrogenation unit to generate a fifth stream comprising butadienes; passing the fifth stream to a butadiene extraction unit; passing the fourth stream to an olefin cracking unit to generate a sixth stream comprising C2 to C4 olefins; passing the sixth stream to fractionation unit to generate a seventh stream comprising C4 and lighter hydrocarbons, and an eight stream comprising C5 and heavier hydrocarbons; passing the seventh stream to the light olefin recovery unit; and passing the eight stream to the olefin cracking unit An embodiment of the invention is one, any or all of prior embodiments in this paragraph up through the fourth embodiment in this paragraph wherein the olefin cracking unit comprises a catalyst selected from the group consisting of a catalyst with a SAPO structure, a catalyst with a ZSM-5 structure, a catalyst with a ZSM-11 structure, and mixtures thereof An embodiment of the invention is one, any or all of prior embodiments in this paragraph up through the fourth embodiment in this paragraph wherein the dehydrogenation unit comprises a catalyst for converting isobutylene to CO2. An embodiment of the invention is one, any or all of prior embodiments in this paragraph up through the fourth embodiment in this paragraph wherein the catalyst is selected from the group consisting of a zinc ferrite catalyst (ZnFe), a bismuth molybdenite catalyst (BiMo), and a mixture of thereof. An embodiment of the invention is one, any or all of prior embodiments in this paragraph up through the fourth embodiment in this paragraph further comprising passing the MTO stream to a quench unit to generate an olefins process stream having reduced water and oxygenate content; and passing the reduced water and oxygenate olefins process stream to a compression unit to generate the MTO stream that has been dewatered and compressed. An embodiment of the invention is one, any or all of prior embodiments in this paragraph up through the fourth embodiment in this paragraph wherein the dehydrogenation unit can be an oxidative dehydrogenation unit or a non-oxidative dehydrogenation unit.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

Other objects, advantages and applications of the present invention will become apparent to those skilled in the art from the following detailed description and drawings.

### BRIEF DESCRIPTION OF THE DRAWINGS

FIG. 1 is an embodiment of the process for producing 1,3 butadiene;

FIG. 2 is a second embodiment of the process for producing 1,3 butadiene;

FIG. 3 is a third embodiment of the process for producing 1,3 butadiene.

### DETAILED DESCRIPTION OF THE INVENTION

The production of butadiene is commonly from a by-product stream that is generated by a steam cracker or a naphtha cracker. The principal products desired from steam crackers or naphtha crackers are light olefins, such as ethylene and propylene. The demand for the products from a steam cracker or a naphtha cracker are increasing, but shifting from one product to increase a second product is not desirable as both products are wanted. It can be seen from the future production of butadiene from cracking units will fall short of demand. Therefore, the source of materials for producing butadiene should come from a different source so as to not decrease the light olefins production. This leads to where can an appropriate source of feed for the production of on-purpose butadiene.

The present invention provides for a solution to the problems of butene shortage and butadiene shortage by coupling a technology which makes a feedstream ideal for on-purpose butadiene production with a dehydrogenation process for the production of butadiene. The process also allows for control of the production of either 1-butene or 1,3-butadiene, with control of recycle and decisions regarding the demand for either 1-butene and 1,3-butadiene.

One method of currently increasing the supply of light olefins is a methanol to olefins (MTO) conversion process. This produces ethylene and propylene, but also produces as a by-product C4 and C5 olefins. The C4 olefins are primarily linear olefins with a small amount of isobutylene. The C5 olefins are more branched, and therefore, the C4 olefins are preferred for conversion to 1,3 butadiene. The butenes are collected and then subjected to dehydrogenation to generate the butadiene.

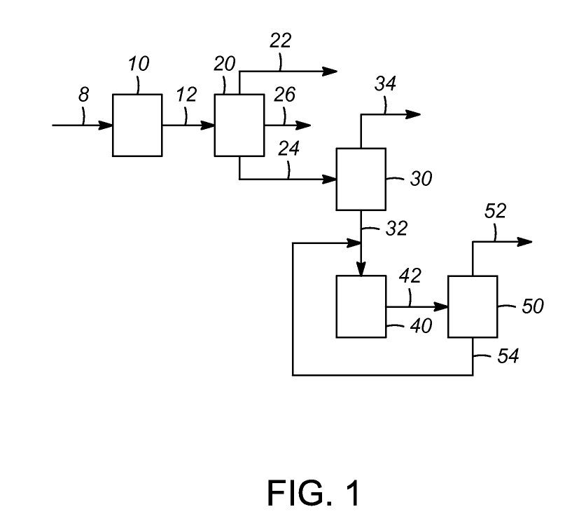

The production of butadiene can be generated from an oxygenate feedstream. The process can be seen in FIG. 1. The oxygenate feedstream 8 is passed to an oxygenate to olefins conversion reactor 10 to generate a process stream 12 comprising olefins, oxygenates and other by-products. The process stream 12 is passed to a first separation unit 20 to generate a first process stream 22, comprising light olefins, and a second process stream 24 comprising C4 and heavier hydrocarbons. The separation unit 20 can also separate residual oxygenates 26 for recycle to the oxygenates to olefins conversion reactor 10. The second process stream 24 is passed to a second separation unit 30 to generate a C4 process stream 32 and a heavies process stream 34. The C4 process stream 32 comprises butenes and butanes, and is passed to a dehydrogenation unit 40 to generate a dehydrogenation process stream 42 comprising butadienes.

The dehydrogenation process stream 42 can be passed to a butadiene extraction unit 50 to generate a butadiene stream 52 comprising 1,3-butadiene, and a second C4 stream 54. The second C4 stream 54 can be passed to the dehydrogenation unit 40 for further conversion of residual C4s. In one embodiment, the process can further include passing the dehydrogenation process stream 42 to a dewatering unit to generate a butadiene stream 52 with a reduced water content. The dewatered stream can be further processed to remove light gases that can be generated in the dehydrogenation unit 40.

In one embodiment of the present invention, the dehydrogenation unit 40 for the dehydrogenation of C4 compounds is an oxidative dehydrogenation unit. The process can further include the removal of isobutylene. Following the separation of butadiene, the second C4 process stream is passed to an isobutylene removal unit. The isobutylene is removed through a reaction to generate a product incorporating the isobutylene and to separate the product from the remaining C4 components, to generate a third process stream having a reduced isobutylene content. The isobutylene removal unit can comprise an MTBE (methyl tertiary butyl ether) or an ETBE (ethyl tertiary butyl ether) unit, where an alcohol, such as methanol or ethanol are fed to the isobutylene removal unit to react and form MTBE of ETBE. Another optional isobutylene removal unit can comprise a tertiary butyl alcohol (TBA) reactor to convert the isobutylene to TBA.

In another embodiment, the process for the production of 1,3 butadiene includes passing an oxygenate stream to an oxygenate conversion reactor to generate a process stream comprising olefins, diolefins and oxygenates. The process includes passing the process stream to a first separation unit to recover the C4s, and thereby generating a light olefins product stream, an oxygenate recycle stream and a C5+ stream. The process includes passing the C4 stream to a butadiene extraction unit to generate a 1,3 butadiene product stream and a second process stream. The second process stream includes butenes and butane, and is passed to a dehydrogenation unit to convert the butenes to butadienes in a dehydrogenated stream. The dehydrogenated stream is passed back to the butadiene extraction unit. The dehydrogenation unit preferably is an oxidative dehydrogenation unit, as it enjoys the benefit of being an exothermic process to facilitate the dehydrogenation reaction.

The process of this embodiment further can include the separation and recovery of 1-butene form the process stream leaving the butadiene extraction unit through a separation unit. The separation unit generates a bottoms stream with the 1-butene removed for passing to a dehydrogenation reactor to increase the 1,3-butadiene content. The process can further include an isobutylene removal unit should the olefin generation process create isobutylene.

The amounts of isobutylene are generally low but need to be removed as the isobutylene will complicate downstream processes. The process can further include a selective hydrogenation step to remove acetylenes generated in the MTO process or in the dehydrogenation process.

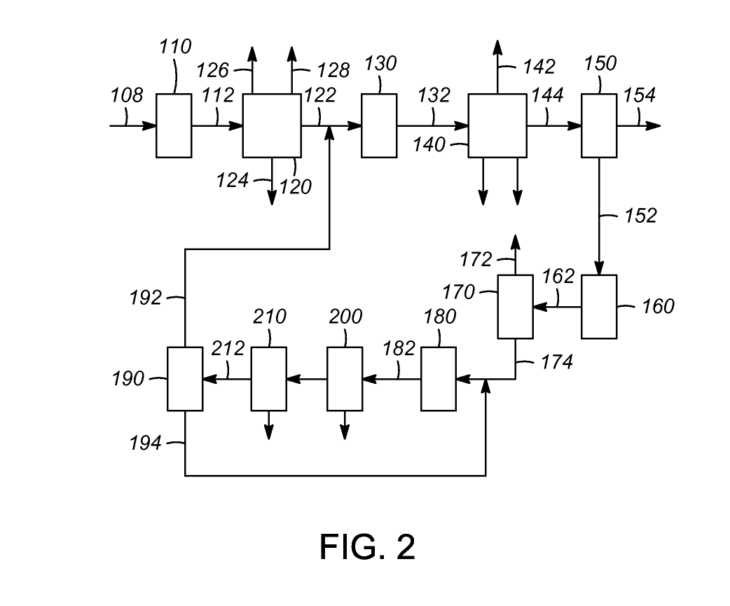

A second embodiment is shown in FIG. 2. The process includes passing an oxygenate stream 108 to an oxygenate conversion reactor 110 to generate a first process stream 112 comprising olefins, diolefins and oxygenates. The first process stream 112 is passed to a first separation unit 120 to generate a light olefins stream 126, a C4+ stream 122 and a recycle stream 124 comprising oxygenates. The separation unit 120 can include operation units for separating any C5+ hydrocarbons to generate a C5+ stream 128. The C4 stream 122 is passed to a selective hydrogenation unit 130 to generate a C4+ stream with a reduced acetylenes content 132. The reduced acetylenes content stream 132 is passed to a butadiene extraction unit 140 to generate a 1,3 butadiene stream 142 and a residual C4 stream 144. The residual C4 144 is passed to an isobutylene removal unit 150 to generate a by-product C4 raffinate stream 152. The by-product C4 raffinate stream 152 is passed to a selective hydrogenation unit 160 to generate a hydrogenated raffinate stream 162. The hydrogenation raffinate stream 162 is passed to a butene separation unit 170 to generate an overhead stream 172 comprising 1-butene and a bottoms stream 174 comprising 2-butene. The bottoms stream 174 is passed to a dehydrogenation unit 180 to generate a dehydrogenated process stream 182 comprising 1,3-butadiene. The dehydrogenation process stream 182 is passed to a second separation unit 190 to generate a second overhead stream 192 comprising 1,3-butadiene, and a second bottoms stream 194. The second overhead stream 192 is passed to the butadiene extraction unit 140.

The process can further include passing the second bottoms stream 194 to the dehydrogenation unit 180. The process can also further include passing the dehydrogenation process stream 182 to a dewatering unit 200, and to a light gas removal unit 210 to generate a process stream 212 having the water and light gases removed.

The 1-butene can be used for a separate product line. The process can further include a separation unit upstream of the butadiene extraction unit 140 to separate the C5+ hydrocarbons from the process stream. This can include having the first separation unit 120 include a C5+ separation section. The isobutylene removal unit 150 can comprise an MTBE (methyl-tertiary butyl ether) reactor for removing isobutylene.

Non-oxidative dehydrogenation is a process for the conversion of hydrocarbons such as alkanes to alkenes. The reaction conditions include operation of the reactor at a temperature between 600° C. and 700° C. The mixing is to provide a more uniform temperature, and it is preferred to sufficiently mix the catalyst and feed to operate at a temperature between 630° C. and 650° C. The reaction conditions include a pressure at the reactor outlet in the rage from 108 kPa to 170 kPa (1 to 10 psig). The preferred operation controls the pressure at the reactor outlet in the range from 122 kPa to 136 kPa (3 to 5 psig). The reaction operates under an atmosphere comprising hydrogen, in addition to the hydrogen generated. The operation of the reactor includes a hydrogen to hydrocarbon mole ratio at the reactor inlet in the range between 0.2 and 1, with a preferred hydrogen to hydrocarbon mole ratio at approximately 0.6.

Any suitable dehydrogenation catalyst may be used in the process of the present invention. Generally, the preferred catalyst comprises a platinum group component, an alkali metal component and a porous inorganic carrier material. The catalyst may also contain promoter metals which advantageously improve the performance of the catalyst. It is preferably that the porous carrier material of the dehydrogenation catalyst be an absorptive high surface area support having a surface are of about 25 to about 500 m2/g. The porous carrier material should be relatively refractory to the conditions utilized in the reaction zone and may be chosen from those carrier materials which have traditionally been utilized in dual function hydrocarbon conversion catalysts. The preferred dehydrogenation catalyst also contains a platinum group component. Of the platinum group metals, which include palladium, rhodium, ruthenium, osmium or iridium, the use of platinum is preferred.

Oxidative dehydrogenation is a catalytic process that is becoming useful for the conversion hydrocarbons in the conversion of hydrocarbon products. One such usage is the dehydrogenation of olefins to diolefins. Low molecular weight hydrocarbons such as ethane and propane are readily transformed by non-oxidative dehydrogenation to generate ethylene and propylene. Oxidative dehydrogenation offers an alternative route, and since the reaction is exothermic, it avoids the thermodynamic constraints of non-oxidative dehydrogenation. This process eliminates carbon deposition on the catalyst, and forms water. This leads to stable activity. The process is limited by the combustion of some of the alkanes to CO and CO2 and also generates water which then leads to the addition of a process for water removal.

Catalytic oxidative dehydrogenation includes a catalyst such as vanadium pentoxide (V2O5), magnesium oxide (MgO) supported vanadium, gamma aluminam supported vanadium.

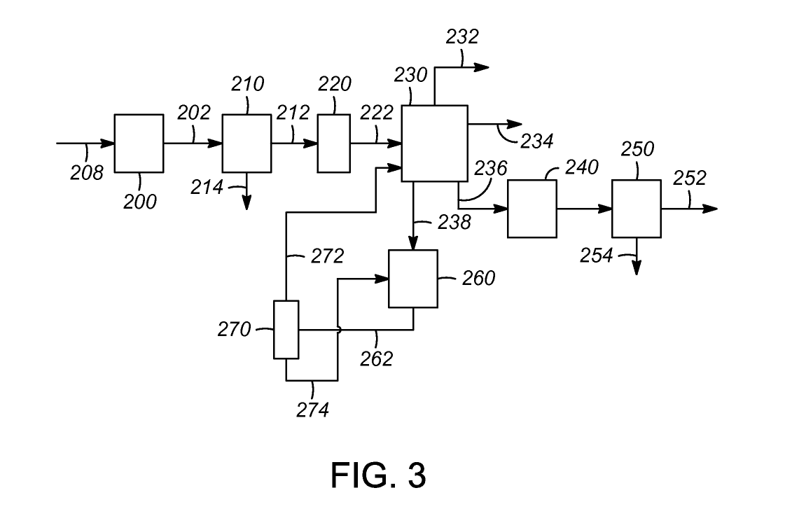

A third embodiment is shown in FIG. 3. The process is for the recovery of 1,3 butadiene. The MTO process generates butenes as a by-product in the production of ethylene and propylene. There is an increasing demand for 1,3 butadiene, and the butenes can be recovered and converted to 1,3 butadiene. In this embodiment, an oxygenate stream 208 is passed to an MTO unit 200 to generate an MTO stream 202 comprising olefins. The oxygenate stream can comprise alcohols, ethers, or other oxygenates, but a preferred stream is methanol or other alcohol. The MTO stream 202 is passed to a quench unit 210 where the MTO stream is cooled and water and a substantial portion of the oxygenates are removed to generate an MTO stream 212 having a reduced water and oxygenate concentration. The water and residual oxygenates are passed out as a process stream 214 for either recycle or further processing. The MTO stream 212 is passed to a compression unit 220 to generate a compressed olefins stream 222. The compressed olefins stream 222 is passed to a light olefins recovery unit 230, or light olefins recovery process (LORP). The light olefins recovery unit 230 comprises a plurality of fractionation columns operated at fractionation conditions to generate the first stream comprising ethylene, the second stream comprising propylene, the third stream comprising butenes, and the fourth stream comprising C5+ hydrocarbons. The light olefins recovery unit 230 can include absorbers, and/or reactors to remove residual undesired compounds. The LORP separates the compressed olefins stream 222 into a first stream comprising ethylene 232, a second stream comprising propylene 234, a third stream 236 comprising butenes, and a fourth stream 238 comprising C5 and heavier hydrocarbons, or C5+ hydrocarbons. The third stream 236 is passed to a dehydrogenation unit 240 to convert the butenes to butadienes, and generates a fifth stream 242 comprising butadienes. The preferred conversion is to convert to 1,3 butadiene. The fifth stream 242 is passed to a butadiene extraction unit 250 to generate the desired 1,3 butadiene product stream 252. The butadiene extraction unit 252 can generate a butane stream 254, which can be purged or passed to downstream process units. The fourth stream 238 can be processed to increase the light olefins products and the butadienes. The fourth stream 238 is passed to an olefins cracking unit 260 to generate a sixth stream 262 comprising C2 to C4 olefins. The sixth stream 262 is passed to a fractionation unit 270 to separate the C4 and lighter components from the heavier hydrocarbons. The fractionation unit 270 generates an overhead stream 272 comprising C4 and lighter hydrocarbons and a bottoms stream 274 comprising C5 and heavier hydrocarbons. The overhead stream 272 is passed to the light olefins recovery unit 230 to remove any C2 to C4 olefins generated. The bottoms stream 274 is passed back to the olefins cracking unit 260 to further crack larger olefins left over from the first pass.

The dehydrogenation unit 250 comprises a catalyst for the dehydrogenation of butenes to butadiene. The catalyst can include a second catalyst for the conversion of isobutylene to CO2. Isobutylene is an undesired impurity for a 1,3 butadiene process stream. The catalyst can be a dehydrogenation catalyst with a dual function for the conversion of isobutylene. Catalysts that can be used include molybdenum-bismuth (MoBi), bi-metallic catalysts that include cobalt (Co), nickel (Ni), or zinc (Zn), and other metals from Groups VIB, VIIB and VIII of the periodic table. One catalyst for use in the dehydrogenation unit is zinc ferrite (ZnFe).

The olefin cracking unit 260 includes a catalyst for cracking the larger olefins to light olefins and butenes. The catalyst can be any olefins cracking catalyst, but a preferred catalyst is a SAPO type catalyst for the generation of linear olefins in the cracking process. This reduces the production of isobutylene.

While the invention has been described with what are presently considered the preferred embodiments, it is to be understood that the invention is not limited to the disclosed embodiments, but it is intended to cover various modifications and equivalent arrangements included within the scope of the appended claims.

What is claimed is:

1. A process for the production of butadiene comprising:
passing a process stream from an oxygenate to olefins reactor to a first separation unit to generate a first process stream comprising light olefins, and a second process stream comprising C4 and heavier olefins;
passing the second process stream to a second separation unit to generate a C4 process stream and a heavies process stream, wherein the C4 process stream comprises butenes and butanes, and an isobutene content of less than 1 wt %; and
passing the butene process stream to a dehydrogenation unit to generate a dehydrogenation process stream comprising butadienes.

2. The process of claim 1 further comprising passing the dehydrogenation process stream to a butadiene extraction unit to generate a butadiene stream comprising 1,3-butadiene, and a second C4 stream.

3. The process of claim 2 further comprising passing the second C4 stream to the dehydrogenation unit.

4. The process of claim 1 further comprising passing the dehydrogenation process stream to a dewatering unit to generate a butadiene stream with reduced water content.

5. The process of claim 4 further comprising passing the dewatered stream to a degassing unit to generate a degassed stream.

6. The process of claim 1 wherein the dehydrogenation unit is an oxidative dehydrogenation unit.

7. A process for the production of 1,3 butadiene, comprising:
passing an oxygenate stream to an oxygenate conversion reactor to generate a process stream comprising olefins, diolefins and oxygenates;
passing the process stream to a first separation unit to generate a light olefins stream, an oxygenate recycle stream, a C5+ stream, and a C4 process stream having an isobutene content of less than 1 wt %;
passing the C4 stream to a butadiene extraction unit to generate a 1,3 butadiene stream and a second process stream;
passing the second process stream to a dehydrogenation unit to generate a dehydrogenated stream; and
passing the dehydrogenated stream to the butadiene extraction unit.

8. The process of claim 7 wherein the dehydrogenation unit is an oxidative dehydrogenation unit.

9. The process of claim 7 further comprising passing the second process stream to a second separation unit to generate a stream comprising 1-butene, and a second C4 stream.

10. The process of claim 7 wherein the dehydrogenation unit is a non-oxidative dehydrogenation unit.

11. A process for the production of 1,3-butadiene, comprising:
passing an oxygenate stream to an oxygenate conversion reactor to generate a first process stream comprising olefins, diolefins and oxygenates;
passing the first process stream to a first separation unit to generate a light olefins stream, a C4 stream and a recycle stream;
passing the C4 stream to a selective hydrogenation unit to generate a C4 stream with reduced acetylenes content;
passing the C4 stream with reduced acetylene content to a butadiene extraction unit to generate a 1,3 butadiene stream, and a residual C4 stream;
passing the byproduct C4 stream to a selective hydrogenation unit to generate a hydrogenated raffinate stream;
passing the hydrogenated raffinate stream to a butene separation unit to generate an overhead stream comprising 1-butene and a bottoms stream comprising 2-butene;
passing the bottoms stream to a dehydrogenation unit to generate a dehydrogenated stream comprising 1,3-butadiene;
passing the dehydrogenated stream to a dewatering unit to generate a dewatered dehydrogenated stream;
passing the dewatered stream to a light gas stripping unit to generate a light end stream and a second process stream comprising 1,3-butadiene;
passing the second process stream to a second separation unit to generate a second overhead stream comprising 1,3-butadiene and a second bottoms stream; and
passing the second overhead stream to the butadiene extraction unit.

12. The process of claim 11 further comprising passing the second bottoms stream to the dehydrogenation unit.

13. The process of claim 11 further comprising:
passing the dehydrogenation process stream to a oxygenate removal unit to generate an overhead stream with reduced oxygenate content; and
passing the overhead stream with reduced oxygenate content to the butadiene recovery unit.

14. The process of claim 11 wherein the first separation unit can include operational units for the separation of C5+ hydrocarbons from the first process stream.

15. A process for the production of 1,3 butadiene comprising:
passing an oxygenate stream to a methanol to olefins unit to generate an MTO stream comprising olefins;
passing the MTO stream comprising C2+ olefins to a light olefin recovery unit to generate a first stream comprising ethylene, a second stream comprising propylene, a third stream comprising butenes, and a fourth stream comprising C5+ hydrocarbons;
passing the third stream to a dehydrogenation unit to generate a fifth stream comprising butadienes;
passing the fifth stream to a butadiene extraction unit;
passing the fourth stream to an olefin cracking unit to generate a sixth stream comprising C2 to C4 olefins;
passing the sixth stream to fractionation unit to generate a seventh stream comprising C4 and lighter hydrocarbons, and an eight stream comprising C5 and heavier hydrocarbons;
passing the seventh stream to the light olefin recovery unit; and
passing the eight stream to the olefin cracking unit

16. The process of claim 15 wherein the olefin cracking unit comprises a catalyst selected from the group consisting of a catalyst with a SAPO structure, a catalyst with a ZSM-5 structure, a catalyst with a ZSM-11 structure, and mixtures thereof

17. The process of claim 15 wherein the dehydrogenation unit comprises a catalyst for converting isobutylene to CO2.

18. The process of claim 17 wherein the catalyst is selected from the group consisting of a zinc ferrite catalyst (ZnFe), a bismuth molybdenite catalyst (BiMo), and a mixture of thereof

19. The process of claim 15 further comprising:
passing the MTO stream to a quench unit to generate an olefins process stream having reduced water and oxygenate content; and
passing the reduced water and oxygenate olefins process stream to a compression unit to generate the MTO stream that has been dewatered and compressed.

20. The process of claim 15 wherein the dehydrogenation unit can be an oxidative dehydrogenation unit or a non-oxidative dehydrogenation unit.
